(19) United States
(12) Patent Application Publication
(10) Pub. No.: US 2022/0111179 A1
(43) Pub. Date: Apr. 14, 2022

(54) RADIAL ARTERY ADAPTOR

(71) Applicant: Cornell University, Ithaca, NY (US)

(72) Inventor: Shing Chiu Wong, Manhasset, NY (US)

(21) Appl. No.: 17/419,150

(22) PCT Filed: Dec. 18, 2019
(86) PCT No.: PCT/US2019/067223

§ 371 Date: Jun. 28, 2021

Related U.S. Application Data
(60) Provisional application No. 62/786,651, filed on Dec. 31, 2018.

(51) Int. Cl. A61M 25/02; A61M 25/00; A61M 25/06

(52) U.S. Cl. CPC A61M 25/02; A61M 25/0097; A61M 2025/0266; A61M 2025/026; A61M 25/06

(57) ABSTRACT

A radial artery catheter adapter comprising, a converter sheath configured to provide access to the radial artery, wherein the converter sheath includes a conduit for a catheter, and the converter sheath is angled around the anatomy such that a first terminus is positioned at least twenty degrees from a substantial portion of the converter sheath a hub for coupling one or more catheters; and an immobilizer configured to stabilize the orientation of the converter sheath and the hub.

### CROSS-REFERENCE TO RELATED APPLICATION

This application claims the benefit of priority to U.S. Provisional Patent Application Ser. No. 62/786,651 entitled “RADIAL ARTERY ADAPTOR,” filed Dec. 31, 2018, the disclosure of which is incorporated herein in its entirety by reference.

### BACKGROUND

An artery access catheter or radial sheath can be used to gain radial arterial access and facilitate the insertion of other equipment for diagnostic and vascular interventions. In some all cases, the patient to which the catheter is inserted, must be immobilized throughout the procedure. Immobilization can cause discomfort to the patient due to the position in which the patient is immobilized resulting in an unnatural posture during the procedure.

### SUMMARY

Disclosed herein is a radial sheath having, for example, an integrated preformed curved shape or an adapter over a conventional (e.g., straight tubular) radial sheath for converting the proximal end of a conventional radial sheath to conform to contour of the wrist artery anatomy to enable supination of the distal upper limb following sheath insertion. The converter sheath can be configured to provide access to a radial artery and simultaneously enable the radial artery access site to be positioned in a pronated position as compared to a supinated position. Furthermore, the pre-shaped radial sheath or the converter sheath can be angled around the anatomy of a patient such that a first terminus of the converter sheath is positioned at least about ten degrees from a substantial proximal portion of the converter sheath. The converter sheath can further include a hub for coupling one or more catheters. The converter sheath can further include an immobilizer configured to stabilize the orientation of the converter sheath and the hub.

### BRIEF DESCRIPTION OF THE FIGURES

The patent or application file contains at least one drawing executed in color. Copies of this patent or patent application publication with color drawing(s) will be provided by the Office upon request and payment of the necessary fee.

In the drawings, which are not necessarily drawn to scale, like numerals describe substantially similar components throughout the several views. The drawings illustrate generally, by way of example, but not by way of limitation, various embodiments discussed in the present document.

FIG. 1 is a top view of a pre-shaped radial artery catheter or a conventional sheath attached to a curved adapter for reshaping the proximal end of sheath outside of the sheath skin insertion site, in accordance with various embodiments.

FIG. 2 is a schematic view of a limb, in accordance with various embodiments.

FIGS. 3A and 3B are respective top and sectional views of flexible inducer, in accordance with various embodiments.

FIG. 4 is a schematic depiction of a tube being positioned in an artery in a compressed state and being expanded once a catheter is in place, in accordance with various embodiments.

FIGS. 5A and 5B are top views of immobilizers, to reshape a conventional catheter to conform of a wrist anatomy once the sheath exits the skin, in accordance with various embodiments.

FIGS. 6A and 6B are top views of a left handed angled line-in port in which a curved introducer sheath or conventional sheath reshaped with a converter after it exits the skin insertion side is oriented at about 45 degrees relative to a catheter, in accordance with various embodiments.

FIG. 7 is a top view of a built-in extender to allow smooth curving in the proximal part of the radial sheath, in accordance with various embodiments.

FIG. 8 is a top view showing a pre-curved catheter or conventional catheter fitted with converter sheath inserted into the right arm of a patient, in accordance with various embodiments.

FIG. 9 is a side view showing a catheter following its insertion into a patient with the proximal extravascular part curved to conform to the wrist in a pronate position, in accordance with various embodiments.

FIG. 10 is a sectional view of a catheter, in accordance with various embodiments.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X. Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

According to various embodiments of the present disclosure, an artery access catheter can be used to gain arterial access and facilitate the insertion of equipment for diagnostic and vascular interventions. There are multiple accessible venues for the artery access catheter, including radial and femoral arteries. Compared with procedures performed via the commonly used femoral artery access route, according to various embodiments, procedures performed via the radial artery route can allow shorter recovery time, earlier ambulation, earlier discharge, and lower incidences of access-site complications, as well as being potentially advantageous for patients with, for example, occlusive aortoiliac disease or peripheral vascular disease. One challenge in using the radial artery route, however, is that the positioning and orientation of the patient's arm with the palm facing palm up, usually in a supination position, can result in discomfort and in some cases, unexpected shift in positioning by the patient. The instant disclosure may provide a solution to this problem. According to some examples, the assemblies described herein are capable of being inserted into the radial artery itself as opposed to the distal radial artery. The can be beneficial because the distal arty is much smaller and is, therefore, a more difficult artery to target.

As an example of a possible solution, according to various embodiments, the instantly described radial artery catheter adapter can be used to position the patient's arm with the palm facing in, towards the body of the patient, in a pronation position. The pronation position, or a partial pronation position can be understood to refer to a position in which is when the palm of the hand is substantially perpendicular to the floor. The adaptors and modifications to the artery catheter described herein are generally configured to correspond, geometrically, to the radial artery, but they can be generalized to catheters for other specific applications. For example, they can also be used for ease of access to other arteries including the femoral or brachial arteries.

FIG. 1 is a top view of a radial artery catheter adapter 101. The adapter 101 includes a converter sheath 105. In some embodiments, the artery catheter adapter can also include an interior sheath 103 to guide a catheter. In addition, the adapter 101 also includes an immobilizer 107 to stabilize the adapter to the anatomy of the patient. The adapter 101 further includes a hub 109. In some embodiments, the adapter further includes a side port 111 for additional access to the conduit of the converter sheath 105.

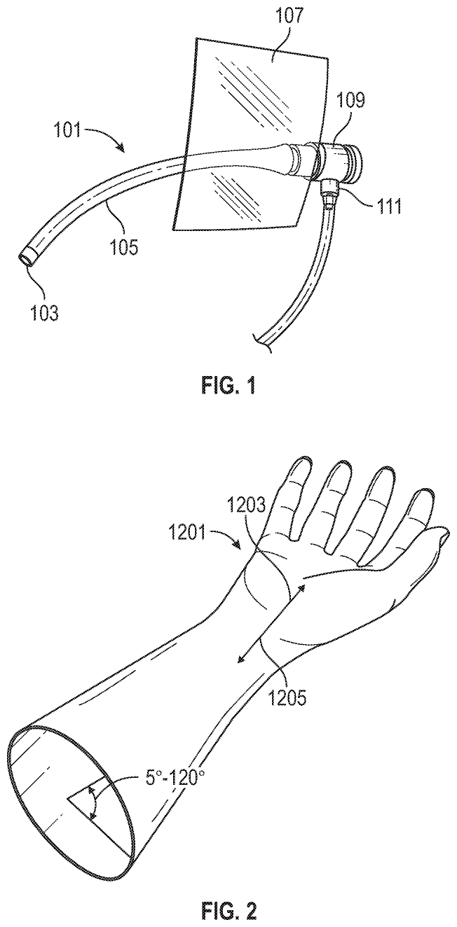

The pre-shaped sheath or conventional sheath fitted with converter 105 includes a conduit for a catheter. The lumen of the conduit can be one of many suitable sizes depending on the desired catheter. For example, a 5 to 7 French catheter can be used. The conduit can be designed to accommodate such sizes specifically or can be specific to a certain catheter size. In general, the lumen of the conduit is in a range of about 0.3 mm to about 3 mm wide, about 0.4 mm to about 2.8 mm, about 0.6 mm to about 2.6 mm, about 0.8 mm to about 2.4 mm, about 1 mm to about 2.2 mm, about 1.2 mm to about 2 mm, about 1.4 mm to about 1.8 mm, less than, equal to, or greater than about 0.3 mm, 0.4.0.5, 0.6, 0.7.0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 mm. The converter sheath 105 has a hub terminus where a hub 109 can be coupled to the sheath and an artery terminus that is configured to align with the artery of interest of the patient. The point of operation by the surgeon, technician or other operator is the hub terminus. The point of entry to the artery of the patient is the artery terminus. In some embodiments of the converter sheath 105, the artery terminus is wrapped around the anatomy of the patient with respect to the hub terminus allowing access to the hub for the operator while the limb of the patient is pronated. The degree to which the termini are offset can vary but according to some embodiments of the adapter, the angle of offset about the limb of the patient can be at least about 1 degree, at least about 5 degrees, at least about 10 degrees, at least about 15 degrees, at least about 20 degrees, at least about 25 degrees, at least about 30 degrees, at least about 35 degrees or more, in a range of from about 1 degree to about 90 degrees, about 5 degrees to about 60 degrees, about 10 degrees to about 30 degrees, as further shown in FIG. 2, which is a schematic view of a limb 1201 of a patent. In some embodiments the termini of the converter sheath can be spaced along the limb of the patient. This spacing (measured between termini 1203 and 1205) can be about 3 cm to about 8 cm, about 3.5 cm to about 7.5 cm, about 4 cm to about 7 cm, about 4.5 cm to about 6.5 cm, about 5 cm to about 6 cm, less than, equal to, or greater than about 3 cm, 3.5, 4, 4.5, 5, 5.5, 6, 6.5, 7, 7.5, or about 8 cm.

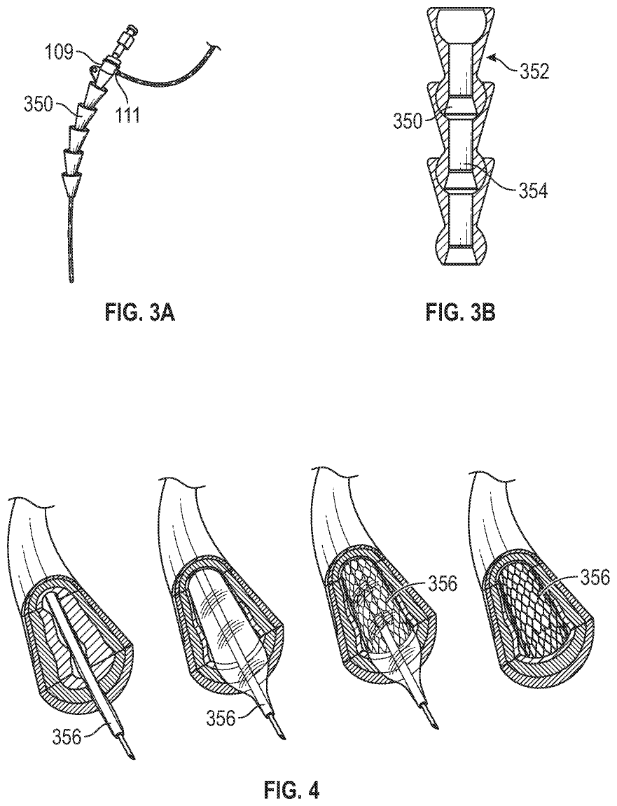

One embodiment of the catheter to be placed in the conduit of the converter sheath 105 is to have a flexible inducer, which will allow the clinician to guide the catheter. Once the catheter has been properly placed, rigidity can be introduced to keep the catheter in place. This is seen in FIGS. 3A and 3B, which are respective top and sectional views of flexible inducer 350. In one embodiment the flexible inducer 350 is a flexible corrugated outer tube 352 with a flexible but stiffened inner tube 354 (inside the corrugated tube), where the far end is guided to the proper placement. Once the far end is placed properly, the near end is secured to stabilize the device as the catheter is inserted through it. Another embodiment is to have a ball and socket design. Once again, when the far end is properly placed, the other end can be pulled to introduce rigidity. A third embodiment is to have a tube 356, that can be positioned in a compressed state and expanded once the catheter is in place. FIG. 4 is a schematic depiction of a tube being positioned in an artery in a compressed state and being expanded once a catheter is in place. In one embodiment of this disclosure, the adaptor 101 initially may be too narrow for the catheter to fit through, but once the adaptor is in place, and the tube expanded, the catheter can be inserted through the tubing and into the artery.

The adapter 101 includes the converter sheath 105 for guiding the catheter to the point of entry into the radial artery of the patient. The converter sheath 105 is a channel that guides the catheter, from the point of operation, to the point of entry into the radial artery of the patient. In general, the point of operation is the side of the adapter 101 that can include the hub 109 and the immobilizer 107 and is the side of the adapter 101 that the operator or surgeon accesses. The point of entry is the other side of the adapter and is aligned to guide the catheter to the radial artery of the patient. The converter sheath 105 includes a conduit for the catheter to pass through. In one embodiment, the converter sheath 105 includes an interior sheath 103. In one embodiment, the interior sheath 103 and converter sheath 105 are integrated into the sheath design. For example, the interior sheath 103 and the converter sheath 105 can be one piece of molded plastic, rubber or other suitable material. In one embodiment, the converter sheath 105 is made of a semi-rigid material and able to retain its shape to conform to the contours of the wrist, palm, or back of hand to which it is placed. As an example, the converter sheath 105 can be made of rubber or soft plastic and can be affixed to the immobilizer 107 at one or more points.

In some embodiments the catheter fits through the converter sheath 105, and the interior sheath 103. Either the converter sheath 105 or the interior sheath 103 can be made from different materials that are formed to have these structures. Because in some embodiments either the converter sheath 105 or the interior sheath 103 can be placed directly under the skin, the materials can be biocompatible, for example the may include a polymer-based product such as an amino acid derived polymer. Additional biocompatible materials can include a bioactive coating that can substantially prevent inflammation, for example polyaspirin. It can also be treated plastic or metal with a bioactive coating, if the material can contour to fill the clinical need it is suitable. The catheter can be made of a soft or flexible material to navigate through the artery. Any variety of commercially available catheters that are suitable for entry into the radial artery can be used. The catheter can pass though the converter sheath 105 into the artery. In one embodiment, the catheter fits through a side port 111 into a hub 109. The hub 109 also includes one or more conduits suitable for the catheter to pass through. Optionally, the catheter also passes through an interior sheath 103 that can be included within the converter sheath 105.

The adapter 101 can exist in more than one size. For example, a one embodiment of the adapter can include the converter sheath 105 having a 90-degree radial curve over about 2.5 cm to about 13 cm, about 5 cm to about 10 cm, about 7 cm to about 8 cm, less than, equal to, or greater than about 2.5 cm, 3, 3.5, 4, 4.5, 5, 5.5, 6, 6.5, 7, 7.5, 8, 8.5, 9, 9.5, 10, 10.5, 11, 11.5, 12, 12.5, or about 13 cm. Another embodiment of the adapter 101 can have a curve of 90-degrees radially over a distance of about 13 cm to about 20 cm, about 15 cm to about 18 cm, less than, equal to, or greater than about 13 cm, 13.5, 14, 14.5, 15, 15.5, 16, 16.5, 17, 17.5, 18, 18.5, 19, 19.5, or about 20 cm. In some embodiments that curve is helical around the hand where the axial distance is from about 2 cm to about 10 cm, about 4 cm to about 8 cm, less than, equal to, or greater than about 2 cm, 2.5, 3, 3.5, 4, 4.5, 5, 5.5, 6, 6.5, 7, 7.5, 8, 8.5, 9, 9.5, or about 10 cm and the curve has a radius length from about 2 cm to about 10 cm, about 4 cm to about 8 cm, less than, equal to, or greater than about 2 cm, 2.5, 3, 3.5, 4, 4.5, 5, 5.5, 6, 6.5, 7, 7.5, 8, 8.5, 9, 9.5, or about 10 cm and an angle from about 1 degree to about 180 degrees, about 5 degrees to about 90 degrees, about 20 degrees to about 180 degrees, about 40 degrees to about 160 degrees, about 60 degrees to about 140 degrees, about 80 degrees to about 120 degrees, about 100 degrees to about 110 degrees, less than, equal to, or greater than about 20 degrees, 30, 40, 50, 60, 70, 80, 90, 100, 110, 120, 130, 140, 150, 160, 170, or about 180 degrees.

The lumen that is within the converter sheath 105 is wide enough for the catheter to pass through. In one variation, the lumen, or conduit, is from about 0.2 mm to about 3 mm wide, about 0.4 mm to about 1.8 mm, about 0.6 mm to about 1.6 mm, about 0.8 mm to about 1.4 mm, about 1 mm to about 1.2 mm, less than, equal to, or greater than about 0.2 mm, 0.3, 0.4, 0.5, 0.6, 0.7, 0.8, 0.9, 1, 1.1, 1.2, 1.3, 1.4, 1.5, 1.6, 1.8, 1.9, or about 3 mm. In some embodiments the conduit is wider than the artery to speed the flow of liquid through the artery. In other embodiments the conduit is smaller than or the same size as the artery. In some embodiments, the width of the conduit is uniform through the side port 111, the hub 109, the immobilizer 107, the converter sheath 105, and the interior sheath 103, in whatever order the subparts are arranged. In some embodiments, varied widths throughout the side port 111, hub 109, immobilizer 107, converter sheath 105, and catheter 301 are desirable.

In some embodiments, the adaptor 101, can include an interior sheath 103 to guide the catheter. The interior sheath 103 is brought with the guide wire 303 into a vein. The interior sheath can have a width from about 0.1 mm to about 1.5 mm, about 0.2 mm to about 1.3 mm, about 0.4 mm to about 1.1 mm, about 0.6 mm to about 0.8 mm, less than, equal to, or greater than about 0.1 mm, 0.2, 0.3, 0.4, 0.5, 0.6, 0.7, 0.8, 0.9, 1, 1.1, 1.2, 1.3, 1.4, or about 1.5 mm. The interior sheath 103 can be tapered along the interior terminus and increase diameter along its length to the exterior terminus with diameters within this range. The interior sheath can function to increase the accessible diameter within the vein to allow the catheter space to fit within the vein. In some embodiments an exterior sheath can also be used to further expand the accessible diameters within the vein, in a range of from about 0.5 mm to about 3 mm, about 0.75 mm to about 2.75 mm, about 1 mm to about 2.5 mm, about 1.25 mm to about 2.25 mm, about 1.5 mm to about 2 mm, less than, equal to, or greater than about 0.5 mm, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 mm. The interior sheath 103 can be made from plastic, medical grade stainless steel, or any other biocompatible, or bioactive material. A consideration in selecting the material may include whether it can expand the vein to allow access to the catheter. The interior sheath 103 is at least partially disposed within the converter sheath 105. The sheaths 103 and 105 can be one unit or they can be separate units. The sheaths 103 and 105 may or may not include the same material or combination of materials. In some embodiments, the interior sheath 103 can be at least partially coated either internally or externally with fluid that can reduce friction between something that needs to be passed either within or outside the interior sheath 103. For example, in some embodiments the guide wire 303 is within the interior sheath and slides along the interior of the interior sheath 103 as it is removed. To assist in the removal, a lubricant can be applied. The lubricant can be bioactive or bioinert. Furthermore, if the interior sheath 103 is removed from within an exterior sheath convertor sheath 105 a lubricant can be used there also.

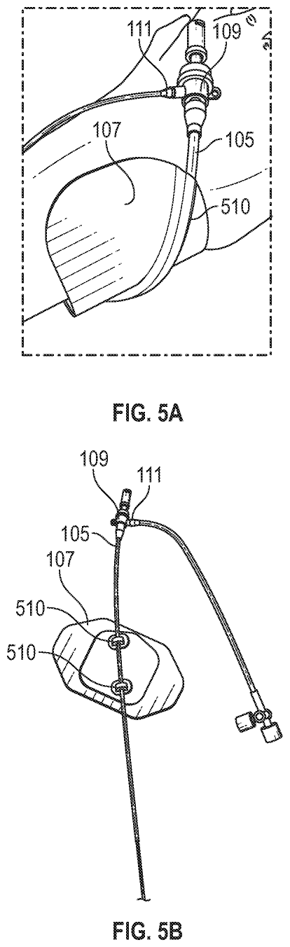

The adapter 101 can also include an immobilizer 107 to prevent displacement of the adapter 101 during or before use. The immobilizer 107 prevents displacement by any one of several suitable mechanisms for stabilizing the position of the adapter 101. For example, in some embodiments, the immobilizer 107 includes a hook-and-loop fastener such as a Velcro™ strap. The Velcro™ strap can be configured to affix the adapter around the hand, wrist or forearm of the patient. The immobilizer 107 can also include tape or adhesive to affix the adapter 101 to the anatomy of the patient. One embodiment of the immobilizer 107 includes an adhesive to secure the converter sheath 105 around the hand of the patient. The adhesive material can wrap around the entire hand of the patient or just a portion of the hand, wrist or forearm of the patient. The immobilizer can be connected directly to a surface that the patient's hand is placed on. For example, the immobilizer 107 can include a brace that is affixed to the surface to hold the converter sheath in place. As mentioned herein, the immobilizer 107 can be integrated into one piece that also includes the converter sheath 105. In further embodiments, the immobilizer 107 can be a separate unit that is coupled to the converter sheath 105. In those embodiments, the immobilizer 107 may be coupled to the converter sheath 105 via a clip, adhesive, Velcro™ or other suitable means. Another embodiment of the immobilizer is a dermal adhesive, such as Tegaderm™, available from 3M Company, MN, as shown in FIGS. 5A and 5B, which are top views of immobilizers. The adhesive can include guides 510 for the catheter to be guided through, allowing for rigid curvature and placement of the catheter.

The adapter 101 may include one or more hubs 109 to allow coupling of one or more catheters, leads or other devices to the adapter 101. The hubs 109 may be separate devices that are attached to the catheter to extend the catheter to allow ease of access. They may also be part of the catheter itself. For example, the adapter 101 can include a hub 109 that includes multiple conduits providing access to the conduit of the converter sheath for multiple catheters. In one embodiment, multiple hubs 109 are integrated into the immobilizer 107 as one piece. In another embodiment, the adapter 101 includes one hub 109 that includes one access point to the conduit of the converter sheath 105. In yet another embodiment, one hub 109 includes multiple access points into the lumen of the converter sheath 105. In another embodiment, multiple separate hubs are coupled to the immobilizer 107 or the converter sheath 105 to allow for multiple access points and venues into the conduit of the converter sheath 105. The hub 109 can also include a diaphragm to prevent foreign matter from going into the sheath, converter sheath 105 or catheter. Also, in some embodiments there can be one or more, side ports attached to the hub. The side ports can be large enough to allow for the catheter to pass through. In addition, they can be configured to carry liquids. In some variations the liquids are of high viscosity like blood. Overall the side ports allow for ease of inserting, or injecting, into sheaths 103 and 105, or the catheter.

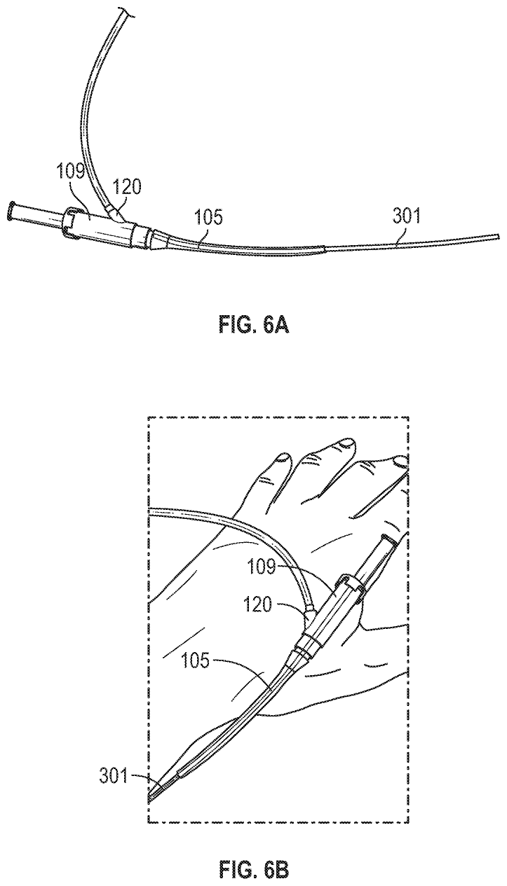

The hub 109 can also include an angled line-in port. The angled line-in port is in a range of from about 1 degree to about 90 degrees, about 30 degrees to about 90 degrees relative to the catheter, about 35 degrees to about 85 degrees, about 40 degrees to about 80 degrees, about 50 degrees to about 70 degrees, less than, equal to, or greater than about 1 degrees, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 degrees. The angled line-in port can be different for a “handed” catheter. This will allow for patient comfort depending on whether the left or right arm is used for access to the artery. This is seen in FIGS. 6A and 6B, which are top views of a left handed angled line-in port where the curved introducer 120 is oriented at about 45 degrees relative to the catheter 301.

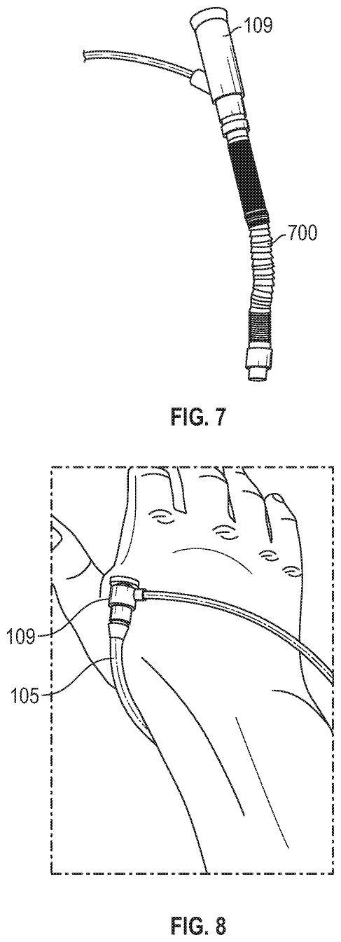

In another embodiment, the radial artery catheter adaptor 101 is an extender 700 of a catheter. FIG. 7 is a top view of the extender 700. The extender 700 can be inserted into the current catheter to extend the catheter in a range of from about 2 cm to about 8 cm, about 3 cm to about 7 cm, about 4 cm to about 6 cm, less than, equal to, or greater than about 2 cm, 2.5, 3, 3.5, 4, 4.5, 5, 5.5.6, 6.5, 7, 7.5, or about 8 cm to allow ease of access and comfort of the patients.

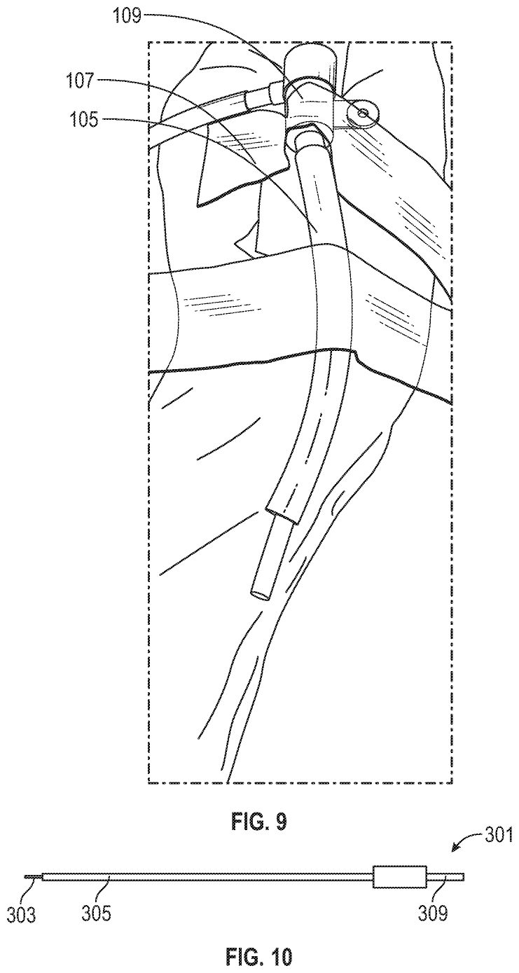

FIG. 8, shows an embodiment of the radial artery catheter adaptor 101 in use. A method of using adaptor 101 includes placing the sheath 103 right arm of the patient. The sheath 103 is immobilized across the abdominal area, which can allow ease of operator access and decreased radiation exposure, if radiation is being injected into the patient. In FIG. 8, the converter sheath 105 can be inserted into the right arm of the patient, and it wraps behind the thumb and with the immobilizer 107 and hub 109 extending towards the palm of the hand. In this embodiment the hand is facing with the thumb flat and the palm is facing the downwards, in a pronation position. As mentioned herein another variation is to have the palm face towards the patient in a partial pronation position. FIG. 8 is for illustrative purposes only, the catheter and converter sheath 105 can wrap around the snuffbox as shown in FIG. 8, but it can be lengthened, and wrapped around the entire hand, for example, or extend further into the palm. One example, of shortening is reaching the snuffbox of the hand, and this is illustrated in FIG. 9 which is a top view, showing the catheter as it is inserted into the patient. Here the immobilizer is tape, but, as mentioned herein, the immobilizer can be anything that holds the converter sheath in place.

FIG. 10 is a sectional view of catheter 301 that can be used in conjunction with adaptor 101. The converter sheath 105 is configured to fit with any commercially available sheath that may be included in catheter 301. Some examples of commercially available sheaths include, the Glidesheath™ radial sheath available from Terumo Interventional Systems, Somerset, N.J.; the Prelude™ radial sheath, available from Meritmedical, South Jordan, Utah; the Cordis Transradial Sheath™, available from Cordis, Milpitas, Calif., and the Newtech clear Sheath™, Haryana, India. The converter sheath 105 can fit over the end of the catheter 301 to allow the sheath 105 to extend to a more comfortable place for the patient. For example, the sheath 105 can extend to the snuffbox of the patient. It can also extend to the palm, forearm, or hand. The extension of the sheath 105 allows the patient to be in a more comfortable position, than supination, for the duration that the catheter is inside the patient. The inner core of the catheter 301 can be removed once the catheter 301 is within the patient. The handle 309 can be pulled once the catheter 301 is within the patient to remove the inner core. In some embodiments, the converter sheath 105 attaches to the catheter 301 within the patient's body. In other embodiments, the converter sheath 105 attaches further away from the patient's body. In some embodiments, the catheter can include an entry needle 305 and a guide wire 303. The entry needle 305 can have a tapered end to allow guide wire 303 introduction into the artery. The entry needle 305, which is hollow, punctures the artery to allow for the guide wire insertion. The entry needle 305 is a trocar and hollow enough minimally to allow for insertion of guide needle 305. The guide wires 303 are designed to navigate vessels to reach a lesion or vessel segment. Once the tip of the device arrives at its destination, it acts as a guide that larger catheters can rapidly follow for easier delivery to the treatment site. The guide wire 303 can be solid steel or nitinol core wires and solid core wire wrapped in a smaller wire coil or braid. A guide wire 303 can be coiled or braided, which offers a large amount of flexibility, pushability and kink resistance. If the wire needs to be seen in an X-ray, common radiopaque markers can be used to improve visibility. These include gold marker bands or the addition of a platinum wire. The configuration of the wire 303 can vary including a “J” curve, a variety of angles or straight tips to help navigate various vessel anatomies. The guide wire 303 diameters can be in a range of from about 0.035 cm to about 0.096 cm, about 0.040 cm to about 0.080 cm, about 0.050 cm to about 0.070 cm, less than, equal to, or greater than about 0.035 cm, 0.040, 0.045, 0.050, 0.055, 0.060, 0.065, 0.070, 0.075, 0.080, 0.085, 0.090, 0.095, or about 0.096. The lengths of the guide wire 303 can range from about 80 cm to about 450 cm, about 100 cm to about 430 cm, about 120 cm to about 410 cm, about 140 cm to about 390 cm, about 160 cm to about 370 cm, about 180 cm to about 350 cm, about 200 cm to about 330 cm, about 220 cm to about 310 cm, about 240 cm to about 280 cm, less than, equal to, or greater than about 80 cm, 90, 100, 110, 120, 130, 140, 150, 160, 170, 180, 200, 210, 220, 230, 240, 250, 260, 270, 280, 290, 300, 310, 320, 330, 340, 350, 360, 370, 380, 390, 400, 410, 420, 430, 440, or about 450 cm.

According to an embodiment of a method of use, described with reference to FIGS. 8-10. The patient can rest their hand on a surface, substantially in a pronation position. The catheter 301 is inserted into the patient over a guidewire through the artery with the guidewire 303. The inner core of the catheter 301 is removed. The converter sheath 105 is then attached to curve the sheath in the direction between thumb and index and so the proximal part of the radial sheath can rest on the “snuff box” area of the pronated hand before the immobilizer 107 is applied, hub 109, and side port 111, is on the end of the radial arterial catheter adaptor 101. The converter sheath 105 or a sheath with a curved proximal pre-shaped curved part extends to the palm of the patient's hand, so the patient can rest their hand in a natural position. The proximal part of an curved sheath of the converter fitted over a conventional sheath 105 is then immobilized via the immobilizer 107, which in this case is Velcro™. The liquids are injected through the side ports 111 that are connected to the hub 109. To remove the catheter 301, the above can be done in reverse. First, the catheter 301 can be unimmobilized by undoing the Velcro™. Then the converter sheath 105 can be removed from the catheter. Then the catheter can be removed from within the patient's arm. Additional Embodiments.

The following exemplary embodiments are provided, the numbering of which is not to be construed as designating levels of importance:

Embodiment 1 provides an artery catheter adapter comprising:
an immobilizer configured to stabilize the artery catheter adapter;
a hub for coupling one or more catheters; and
a converter sheath, comprising:
   an artery terminus configured to align with an entry point to an artery,
   a hub terminus coupled to the hub, and
   a catheter conduit between the artery terminus and the hub.

Embodiment 2 provides the artery catheter adapter of Embodiment 1, wherein the artery terminus of the converter sheath is aligned at an at least one-degree angle from the hub terminus of the converter sheath.

Embodiment 3 provides the artery catheter adaptor of any one of Embodiments 1 or 2, wherein the converter sheath is configured to contour about a limb.

Embodiment 4 provides the artery catheter adaptor of any one of Embodiments 1-3, wherein the converter sheath comprises a flexible corrugated tube.

Embodiment 5 provides the artery catheter adaptor of any one of Embodiments 1-4, wherein the immobilizer further comprises a hook-and-loop fastener to stabilize the artery catheter adapter.

Embodiment 6 provides the artery catheter adaptor of any one of Embodiments 1-5, wherein the artery is a radial, brachial or femoral artery.

Embodiment 7 provides the artery catheter adaptor of any one of Embodiments 1-6, wherein the converter sheath has a length in a range of from about 2 cm to about 8 cm.

Embodiment 8 provides the artery catheter adaptor of any one of Embodiments 1-7, wherein the immobilizer and hub are one unit.

Embodiment 9 provides the artery catheter adaptor of any one of Embodiments 1-8, wherein the immobilizer is an adhesive.

Embodiment 10 provides an assembly comprising:
an immobilizer configured to stabilize the artery catheter adapter;
   a hub for coupling one or more catheters; and
   a converter sheath, comprising:
      an artery terminus configured to align with an entry point to an artery,
      a hub terminus coupled to the hub, and
      a catheter conduit between the artery terminus and the hub; and
one or more catheters coupled to the hub.

Embodiment 11 provides the assembly of Embodiment 10, further comprising an angled line-in port that is in a range of from about 1 degree to about 90 degrees relative to the one or more catheters.

Embodiment 12 provides the assembly of any one of Embodiments 10 or 11, wherein the artery catheter adaptor is an extender coupled to a catheter and adapted to extend the catheter from about 2 cm to about 8 cm.

Embodiment 13 provides the assembly of any one of Embodiments 10-12, wherein the artery terminus of the converter sheath is aligned at an at least one-degree angle from the hub terminus of the converter sheath.

Embodiment 14 provides the assembly of any one of Embodiments 10-12, wherein the converter sheath is configured to contour about a limb.

Embodiment 15 provides the assembly of any one of Embodiments 10-12, wherein the artery is a radial, brachial or femoral artery.

Embodiment 16 provides a method of using an artery catheter adaptor method comprising:
an immobilizer configured to stabilize the artery catheter adapter;
   a hub for coupling one or more catheters; and
   a converter sheath, comprising:
      an artery terminus configured to align with an entry point to an artery,
      a hub terminus coupled to the hub, and
      a catheter conduit between the artery terminus and the hub; and
attaching the artery catheter adaptor to a patient such that the patient's hand can be in a substantially pronated position.

Embodiment 17 provides the method of Embodiment 16, wherein the artery terminus of the converter sheath is aligned at an at least twenty-degree angle from the hub terminus of the converter sheath.

Embodiment 18 provides the method of any one of Embodiments 16 or 17, wherein the converter sheath is configured to contour about a limb.

Embodiment 19 provides the method of any one of Embodiments 16-18, wherein the artery is a radial, brachial or femoral artery.

Embodiment 20 provides the method of any one of Embodiments 16-19, wherein the immobilizer is an adhesive.

1. An artery catheter adapter comprising:
an immobilizer configured to stabilize the artery catheter adapter;
a hub for coupling one or more catheters; and
a converter sheath, comprising:
an artery terminus configured to align with an entry point to an artery,
a hub terminus coupled to the hub, and
a catheter conduit between the artery terminus and the hub.

2. The artery catheter adapter of claim 1, wherein the artery terminus of the converter sheath is aligned at an at least one-degree angle from the hub terminus of the converter sheath.

3. The artery catheter adaptor of any one of claim 1, wherein the converter sheath is configured to contour about a limb.

4. The artery catheter adaptor of claim 1, wherein the converter sheath comprises a flexible corrugated tube.

5. The artery catheter adaptor of claim 1, wherein the immobilizer further comprises a hook-and-loop fastener to stabilize the artery catheter adapter.

6. The artery catheter adaptor of claim 1, wherein the artery is a radial, brachial or femoral artery.

7. The artery catheter adaptor of claim 1, wherein the converter sheath has a length in a range of from about 2 cm to about 8 cm.

8. The artery catheter adaptor of claim 1, wherein the immobilizer and hub are one unit.

9. The artery catheter adaptor of claim 1, wherein the immobilizer is an adhesive.

10. An assembly comprising:
an immobilizer configured to stabilize the artery catheter adapter;
a hub for coupling one or more catheters; and
a converter sheath, comprising:
an artery terminus configured to align with an entry point to an artery,
a hub terminus coupled to the hub, and
a catheter conduit between the artery terminus and the hub; and
one or more catheters coupled to the hub.

11. The assembly of claim 10, further comprising an angled line-in port that is in a range of from about 1 degree to about 90 degrees relative to the one or more catheters.

12. The assembly of claim 10, wherein the artery catheter adaptor is an extender coupled to a catheter and adapted to extend the catheter from about 2 cm to about 8 cm.

13. The assembly of claim 10, wherein the artery terminus of the converter sheath is aligned at an at least one-degree angle from the hub terminus of the converter sheath.

14. The assembly of claim 10, wherein the converter sheath is configured to contour about a limb.

15. The assembly of claim 10, wherein the artery is a radial, brachial or femoral artery.

16. A method of using an artery catheter adaptor method comprising:
an immobilizer configured to stabilize the artery catheter adapter:
a hub for coupling one or more catheters; and
a converter sheath, comprising:
an artery terminus configured to align with an entry point to an artery,
a hub terminus coupled to the hub, and
a catheter conduit between the artery terminus and the hub; and
attaching the artery catheter adaptor to a patient such that the patient's hand can be in a substantially pronated position.

17. The method of claim 16, wherein the artery terminus of the converter sheath is aligned at an at least one-degree angle from the hub terminus of the converter sheath.

18. The method of claim 16, wherein the converter sheath is configured to contour about a limb.

19. The method of claim 16, wherein the artery is a radial, brachial or femoral artery.

20. The method of claim 16, wherein the immobilizer is an adhesive.
